(19) United States
(12) Patent Application Publication
(10) Pub. No.: US 2002/0131943 A1
(43) Pub. Date: Sep. 19, 2002

(54) Hair cap with conditioner or other hair conditioning agent

(76) Inventor: Gary Grey, New York, NY (US)

Correspondence Address: OSTROLENK FABER GERB & SOFFEN, 1180 AVENUE OF THE AMERICAS, NEW YORK, NY, 100368403

(21) Appl. No.: 10/144,412

(22) Filed: May 9, 2002

Related U.S. Application Data
(63) Continuation-in-part of application No. 10/053,126, filed on Jan. 21, 2002.
(60) Provisional application No. 60/269,171, filed on Feb. 15, 2001.

(51) Int. Cl. A61K 7/06; B32B 27/12

(52) U.S. Cl. 424/70.1; 442/123

(57) ABSTRACT

A fabric having a sorbed hair conditioner is provided. The fabric may be knit or woven on non-woven. The conditioner and fabric are such that the conditioner transfers to the hair when the fabric is worn, e.g. as a bonnet. Transfer to the hair may be by any of several techniques, including ionic transfer, heat transfer, use of thixotropic, or non-polar, or hydroscopic conditioning agents. The conditioners maybe of various types, including normal hair conditioners, hair growth agent or hair breakage protection, anti-dandruff or anti-itch agent, fragrance application, hair color protection or others.

### CROSS REFERENCE TO RELATED APPLICATIONS

This is a continuation in part of U.S. Patent Application No. 10/053,126 filed Jan. 21, 2002. That application claims the benefit and priority of U.S. Provisional Application No. 60/269,171 filed Feb. 15, 2001 and entitled “HAIR COVERING WITH CONDITIONER”, and which is incorporated herein by reference.

### BACKGROUND OF THE INVENTION

Various types of hair conditioning compositions are well known.

Several types of conditioner are intended to cause the hair to be soft, manageable and shiny. They also ease the ability to comb hair whether wet or dry, as well as preventing a condition known as “fly-away” of dry hair.

The application of most hair conditioning compositions is on wet hair, usually after the hair has been shampooed. More recently, so called two-in-one conditioning shampoos have been developed which allow a single application and provide both cleansing and conditioning actions in a single preparation.

Hair conditioners can generally be divided into two types. One is referred to in the art as “rinse off” because the conditioner is allowed to remain in contact with the hair for a brief period of time and is then removed by, for instance, rinsing. The other type is called a “leave on” product and this denotes a composition which is applied to the hair, whether in a wet or dry condition, and is not subsequently rinsed off. The “leave on” hair conditioners are typically applied to the hair from a pump-type spray dispenser from which the conditioner emerges either as a mist or a liquid stream.

Berryhill, et al. U.S. Pat. No. 3,088,469 discloses a hair conditioner and retainer with a rectangular swatch of a non-woven fabric that has lanolin applied thereto. The lanolin is preferably anhydrous and is applied to the non-woven by being divided into a fine mist in an ionization chamber, the particles of the mist being charged electrically and caused to precipitate by imposing an intense electrostatic field on the fabric.

There is a need for a hair covering with conditioner(s) which is easy to make and is also easy to use.

### SUMMARY OF THE INVENTION

The present invention provides a hair covering with selected conditioners which can be transferred to a person's hair. More particularly, a woven fabric having a sorbed hair conditioner is provided. The fabric is dry to the touch and does not appear to be wet or greasy. The hair conditioner can be easily desorbed from the fabric and transferred to the hair.

Various types of hair conditioning agents and treatments may be applied to the hair. One type is a standard hair conditioner. A second type includes hair growth promotion treatments, which may include treatments that prevent breaking of the hair and thereby promotes hair growth. A third type might be an anti-itch treatment. A fourth type might be an anti-dandruff treatment. A fifth type might transfer fragrance to the hair. A sixth type protects color treated hair and makes color treatment last longer.

Other features and advantages of the present invention will become apparent from the following description of the invention.

### DESCRIPTION OF THE INVENTION

In accordance with the present invention, a woven or non-woven, or preferably a knit fabric, preferably of a polyester or satin material and having one or more sorbed hair conditioners is provided. The shape and style of the fabric is not restricted and it can be in the form of a bonnet type cap (similar to a shower cap) or a wrap which can be either tied or secured with hook and loop closures.

Any known hair conditioner or combination of conditioners can be employed in the present invention. It or they can be absorbed or adsorbed to the fabric by a variety of approaches. The only requirement of the sorbtion process is that it does not fix the conditioner to the fabric and allows the conditioner to be easily desorbed from the fabric and transferred to the hair.

One type of hair conditioner which can be used is the type containing a positive ionic charge. It is well known that adult human hair, especially hair in need of conditioning, carries a negative ionic charge and positively charged conditioner material is attracted to the negative sites on or in the hair and attach themselves thereto, thereby providing a conditioning effect. These cationic conditioning agents can be sorbed to a sleep cap, for example, and will come into contact with the hair while the person is sleeping.

A second type of hair conditioner is the heat activated conditioner. The heat which activates these materials can be the body's natural warmth or an applied heat from, for example, a blow dryer. The heat, combined with the friction of the hair on the treated fabric causes the conditioner(s) to deposit in or on the hair and provide a conditioning effect.

A third type of conditioner is a thixotropic material. These are conditioners which respond to warmth or body heat by becoming less viscous. As a result, it becomes easier to separate the conditioners from the fabric and transfer them to the hair by way of friction.

A fourth approach involves the use of a non-polar conditioner which is dissolved or suspended in a polar carrier such as an alcohol, water or a combination of water and alcohol, applied to the fabric and allowed to dry. This results in the deposit of a non-greasy hydrophobic material on and in the fabric. Friction between the fabric and the hair causes the transfer of the conditioning agents to the hair.

A fifth type of conditioner uses a hydroscopic conditioning agent which has known humectant, emollient and film-forming properties. The hydroscopic nature of this type of conditioner works to retain moisture and also the moisture balance of the human hair. Transfer from the fabric to the hair is the result of friction with the natural moisture content of the human hair assisting in the transfer.

Any one of the above described types of conditioning agents or treatments may be transferred by any of the techniques described just above. For example, a treated satin or other fabric cap worn during the night may promote hair growth, particularly by preventing breakage of hairs. A hair growth promoter under the made name Trichogen T VEG LS 8960 from Laboratories Serobiologiques is one non-limiting example.

Another example of a conditioner includes a treatment that counteracts itching and/or dandruff. A satin or fabric cap or wrap provided with an appropriate conditioner promotes a moisturized scalp and relief of itching, and it may be worn at night.

Yet another conditioner, might be one that protects color treatment in the hair and causes the color treatment to last a longer time. This might include addition of Heliogenol, a color preservation agent from Sederma.

The hair may be provided with aroma, by a conditioning agent or along with another conditioning agent, so that fragrance is supplied e.g. as the hair is conditioned. There could be ionic transfer of the fragrance from the fabric to the hair or a body heat activated transfer. This method of transferring fragrance has the additional benefit of controlled release of the fragrance, so as not to overwhelm the wearer of the fabric with aroma or not to overwhelm others as well as the wearer after the fabric is removed. These benefits are compared with drawbacks of simply applying a perfume directly to the hair. The fabric may include an odor absorber along with the fragrance applying material.

A hair covering of the present invention can be easily prepared. The following is an example of a process which can be employed using one type of conditioner.

A solution or mixture containing a combination of three conditioners, Dow Silicone 200/200, PEG 45 palm kernel glyceride and a hydrolyzed wheat protein with a silicone backbone (the latter being a heat-activated conditioner) is prepared using a combination of deionized water and isopropyl alcohol as a solvent, glycerine as a humectant, triethanolamine as an agent to adjust the pH to a value appropriate for contact with the hair as well as conventional anti-bacterial agents/preservatives, fragrance and anti-foaming agent, e.g., one obtained from Dow, Antifoam 1410.

The solution was transferred to a 100 denier single jersey knit polyester which had previously been knit, dyed and finished. The solution was sprayed on the fabric causing the aqueous solution of the conditioner to be coated on the fabric and causing it to become wet, i.e., slightly saturated with the mixture of conditioning agent and solvent.

The fabric was then re-rolled onto plastic cores.

The fabric was then placed in a well-ventilated chamber through which air was circulated at a rapid rate for 24 to 48 hours. The water and alcohol solvents evaporated and were removed from the chamber by being entrained in the air leaving the chamber, leaving the conditioning agents on and in the fabric. The resulting fabric containing the sorbed conditioners was dry to the touch.

It will be appreciated that various changes and modifications can be made to the product and process of this invention without departing from the spirit and scope thereof. The various embodiments which have been described herein were set forth in order to further illustrate the invention but were not intended to limit it.

What is claimed is:

1. A fabric having a sorbed hair conditioner, the fabric being dry to the touch.

2. The fabric of claim 1 wherein the fabric is a knitted, woven or non-woven fabric.

3. A fabric according to claim 1 in which the hair conditioner is a cationic hair conditioner.

4. A fabric according to claim 1 in which the hair conditioner is a heat-activated hair conditioner.

5. A fabric according to claim 1 in which the hair conditioner is a thixotropic hair conditioner.

6. A fabric according to claim 1 in which the hair conditioner is a non-polar hair conditioner.

7. A fabric according to claim 1 in which the hair conditioner is a hydroscopic hair conditioner.

8. A fabric according to claim 1 in which a plurality of the hair conditioners are sorbed.

9. A fabric according to claim 1, wherein the hair conditioner is a conditioning agent.

10. A fabric according to claim 1, wherein the hair conditioner is a hair growth promotion treatment.

11. A fabric according to claim 1, wherein the hair conditioner is a dandruff treatment.

12. A fabric according to claim 1, wherein the hair conditioner is an anti-itch treatment.

13. A fabric according to claim 1, wherein the hair conditioner is a fragrance transfer agent.

14. A fabric according to claim 1, wherein the hair conditioner is an antibacterial agent.

15. A fabric according to claim 1, wherein the hair conditioner is a color treatment protection.

16. A fabric according to claim 1 in the form of a wrap or bonnet for the hair.

17. A fabric according to claim 1 in the form of a wrap or bonnet for the hair.

18. A fabric according to claim 1, wherein the conditioner comprises a solution or mixture containing a combination of three conditioners, Dow Silicone 200/200, PEG 45 palm kernel glyceride and a hydrolyzed wheat protein with a silicone backbone (the latter being a heat-activated conditioner), a combination of deionized water and isopropyl alcohol as a solvent, glycerine as a humectant, triethanolamine as an agent to adjust the pH to a value appropriate for contact with the hair and antibacterial agents/preservatives, fragrance and anti-foaming agent.

19. A method of making the fabric having a sorbed conditioner, the fabric being dry to the touch, the method comprising sorbing a hair conditioner to a woven fabric and drying the resulting fabric until it is dry to the touch.

20. The method of claim 19 in which the woven fabric is a knitted fabric.

21. The method of claim 20 in which a solution of a hair conditioner is applied to the fabric followed by causing the solvent of the solution or dispersion to be evaporated and removed from the fabric.

22. The method of claim 21 in which the solution contains a combination of at least two hair conditioners.

23. The method of claim 22 in which at least one of the conditioners is heat activated.

24. The method of claim 19, wherein the conditioner comprises a solution or mixture containing a combination of three conditioners, Dow Silicone 200/200, PEG 45 palm kernel glyceride and a hydrolyzed wheat protein with a silicone backbone (the latter being a heat-activated conditioner), a combination of deionized water and isopropyl alcohol as a solvent, glycerine as a humectant, triethanolamine as an agent to adjust the pH to a value appropriate for contact with the hair and anti-bacterial agents/preservatives, fragrance and anti-foaming agent.
